(19) United States
(12) Patent Application Publication
(10) Pub. No.: US 2023/0165465 A1
(43) Pub. Date: Jun. 1, 2023

(54) REMOTE BODY TEMPERATURE MONITORING SYSTEM

(71) Applicant: AMOSENSE CO.,LTD, Cheonan-si (KR)

(72) Inventor: Jin Sung YANG, Cheonan-si (KR)

(21) Appl. No.: 17/997,405

(22) PCT Filed: Apr. 21, 2021
(86) PCT No.: PCT/KR2021/005048

§ 371 Date: Oct. 28, 2022

(30) Foreign Application Priority Data
Apr. 28, 2020 (KR) 10-2020-0051378

(51) Int. Cl. A61B 5/00; A61B 5/01; H04W 4/14; H04W 4/80

(52) U.S. Cl. CPC A61B 5/0022; A61B 5/01; A61B 5/6833; A61B 5/002; A61B 5/74; A61B 5/0008; H04W 4/14; H04W 4/80; A61B 2562/0271; A61B 2562/164

(57) ABSTRACT

A remote body temperature monitoring system is provided. A remote body temperature monitoring system according to an embodiment of the present invention comprises: a patch-type thermometer attached to the body of a user so as to periodically measure body temperature; a web service server for collecting information about the body temperature measured by the patch-type thermometer and notifying a manager terminal of a suspected fever; a collection device for receiving the information about the measured body temperature from the patch-type thermometer and transmitting same to the web service server; and an AI server for receiving the information about the body temperature from the web service server to manage a health status by using artificial intelligence.

### TECHNICAL FIELD

The present invention relates to a remote body temperature monitoring system.

### BACKGROUND ART

Recently, a viral infection has spread all over the world. Since one of initial symptoms of such an infection is fever, the simplest and most frequently used method of testing for infection is a method of testing a body temperature.

In addition, since such an infection has an incubation period of about two weeks, it is necessary to check a body temperature for a certain period even if a specific symptom does not appear. In particular, there is a need for an effective method of monitoring a body temperature of inbound travelers arriving from countries with a high incidence rate.

Related Document: KR 1729327 B1

### DISCLOSURE

### Technical Problem

The present invention is directed to providing a remote body temperature monitoring system capable of periodically and remotely measuring a body temperature of a monitoring target for a certain period of time.

### Technical Solution

According to an embodiment of the present invention, a remote body temperature monitoring system includes a patch-type thermometer attached to a user's body to periodically measure a body temperature, a web service server configured to collect body temperature information measured by the patch-type thermometer and provide a notification to a manager terminal when a fever is suspected, a collection device configured to receive the measured body temperature information from the patch-type thermometer and transmit the received body temperature information to the web service server, and an artificial intelligence (AI) server configured to receive the body temperature information from the web service server and manage a health status through AI.

The collection device may include an echo device configured to communicatively interwork with the AI server and capable of voice recognition, the patch-type thermometer may periodically transmit data, and the echo device may upload the data received from the patch-type thermometer to the web service server.

The AI server may provide health check guide information according to heat detection to the echo device, and the echo device may output the health check guide information as a voice.

The collection device may include a beacon scanner installed at an entrance of a specific area. The specific area may be a quarantine desk of an airport and a port.

The collection device may include a portable terminal which is Bluetooth-paired with the patch-type thermometer to receive the measured body temperature information and on which an application is installed.

The manager terminal may provide a health check guide according to the notification from the web service server to the portable terminal, and the portable terminal may make an inquiry about the health check guide to the manager terminal.

The web service server may notify the manager terminal through a push message or a short message service (SMS) message.

The patch-type thermometer may include at least one temperature sensor mounted on one surface of a flexible circuit board, a memory configured to store information obtained through the temperature sensor, a communicator configured to transmit the information stored in the memory to an external device, a controller configured to control operation of the temperature sensor, the memory, and the communicator, a power supply disposed on one surface of the flexible circuit board to provide operating power, and a protective member configured to surround the flexible circuit board to prevent external exposure of the temperature sensor, the memory, the communicator, the controller, and the power supply.

### Advantageous Effects

According to the present invention, a body temperature of a monitoring target is periodically and remotely measured for a certain period of time, thereby accurately monitoring the body temperature information of an inbound traveler suspected of having an infection with high reliability.

### DESCRIPTION OF DRAWINGS

FIG. 1 is a schematic block diagram of a remote body temperature monitoring system according to an embodiment of the present invention.

FIG. 2 is a view illustrating a patch-type thermometer of the remote body temperature monitoring system according to an embodiment of the present invention.

FIG. 3 is a bottom view illustrating a state in which a release film is separated in FIG. 2.

FIG. 4 is a view illustrating an internal configuration of FIG. 2.

FIG. 5 is a cross-sectional view along line A-A′ of FIG. 4.

FIG. 6 is a block diagram illustrating the patch-type thermometer of FIG. 2.

### MODES OF THE INVENTION

Hereinafter, embodiments of the present invention will be described in detail with reference to the accompanying drawings so as to be easily practiced by a person of ordinary skill in the art to which the present invention pertains. It should be understood that the present invention may be embodied in various different forms and is not limited to the following embodiments. Parts irrelevant to description are omitted in the drawings in order to clearly describe the present invention, and like reference numerals refer to like elements throughout the specification.

A remote body temperature monitoring system 10 according to an embodiment of the present invention is a body temperature monitoring system using artificial intelligence (AI). In particular, the remote body temperature monitoring system 10 may remotely monitor a body temperature of an inbound traveler suspected of having an infection.

Thus, the remote body temperature monitoring system 10 according to the embodiment of the present invention can accurately monitor a body temperature information for a specific subject, such as an inbound traveler, with high reliability for a certain period of time. Here, the inbound traveler may be a user who is requested to be quarantined for a certain period of time.

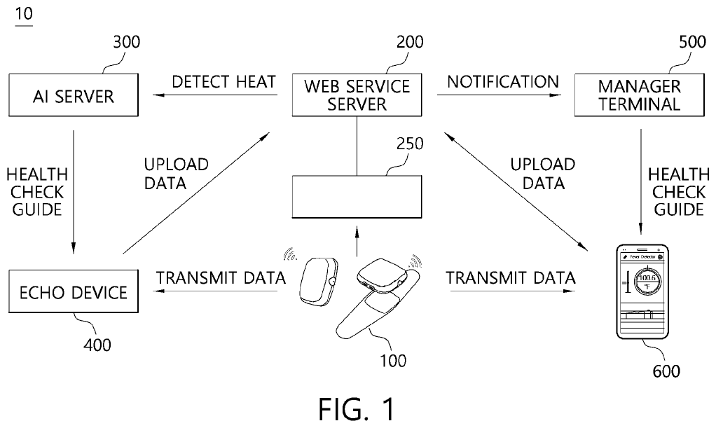

To this end, as shown in FIG. 1, the remote body temperature monitoring system 10 according to the embodiment of the present invention includes a patch-type thermometer 100, a web service server 200, an AI server 300, an echo device 400, a manager terminal 500, and a portable terminal 600.

The patch-type thermometer 100 may be implemented in the form of a patch and attached to a user's body. In a state of being attached to the user's body, the patch-type thermometer 100 may periodically measure a body temperature to then store measurement results. Here, a user may be a monitoring target such as an inbound traveler.

Accordingly, a user can easily check stored body temperature information, thereby easily checking a trend of a change in body temperature of the user.

The web service server 200 may collect body temperature information measured by the patch-type thermometer 100 through a collection device. Here, the collection device may include a beacon scanner 250, the echo device 400, and the portable terminal 600 to be described below.

In addition, the web service server 200 may be a cloud platform and may provide an infrastructure such as a computer, storage, or database. In addition, the web service server 200 may perform AI analysis and a data lake analysis through the AI server 300 to be described below.

As an example, the web service server 200 may request the AI server 300 to detect heat based on body temperature data collected from the patch-type thermometer 100. Here, the web service server 200 may provide information about a trend of a change in body temperature collected from the patch-type thermometer 100 to the AI server 300.

In addition, the web service server 200 may provide a notification to the manager terminal 500 when a monitoring target is suspected of having a fever according to the collected body temperature information. In this case, the web service server 200 may notify the manager terminal 500 through a push message or a short message service (SMS) message.

The AI server 300 may receive body temperature information from the web service server 200 and may determine whether a monitoring target has a fever through AI. In this case, the AI server 300 may determine whether the monitoring target has a fever based on body temperature information of the monitoring target, in particular, a trend of a change in body temperature. For example, in the case of the patch-type thermometer 100, measured body temperature information may be relatively inaccurate, unlike a conventional non-contact type thermometer, and the AI server 300 may infer a health status of a monitoring target through a trend of a change amount A of a body temperature value input from the patch-type thermometer 100. That is, since a change amount of a body temperature value can be monitored even when there is a difference between a measured temperature and an actually measured body temperature, it is possible to identify that a body temperature is abruptly increased to a certain level or more or is repeatedly increased and decreased due to a fever reducer or the like that is taken.

In addition, the AI server 300 may manage a health status based on body temperature information of a monitoring target, in particular, a trend of a change in body temperature in addition to simple fever. That is, the AI server 300 may provide necessary information according to the health status of the monitoring target.

In this case, the AI server 300 may provide health check guide information according to heat detection to the echo device 400. As an example, the AI server 300 may provide health check guide information for guidance and measures according to a change in body temperature of a monitoring target to the echo device 400, which communicatively interworks therewith. However, the AI server 300 may provide such health check guide information directly to the echo device 400 as shown in FIG. 1 or may provide such health check guide information to the echo device 400 through the web service server 200 unlike that shown in FIG. 1.

As another example, the AI server 300 may provide the health check guide information to the manager terminal 500 and the portable terminal 600 through the web service server 200. That is, the web service server 200 may notify the manager terminal 500 or the portable terminal 600 of a health check guide provided from the AI server 300.

The AI server 300 may analyze a health status of a monitoring target according to a request from the web service server 200 or a request from the echo device 400.

The beacon scanner 250, the echo device 400, and the portable terminal 600 may be collection devices according to installation purposes and functions. In this case, the collection device may receive measured body temperature information from the patch-type thermometer 100 to transmit the measured body temperature information to the web service server 200.

Thus, in the remote body temperature monitoring system 10 according to the embodiment of the present invention, information body temperature information of a monitoring target can be collected in various forms, thereby improving reliability in collecting body temperature information of a user who is being quarantined or monitored.

The beacon scanner 250 may be installed at an entrance of a specific area to receive body temperature information transmitted from the patch-type thermometer 100. Here, the specific area may be a quarantine desk of an airport and a port.

Thus, in the remote body temperature monitoring system 10 according to the embodiment of the present invention, since it is possible to easily check whether a monitoring target has a fever through body temperature measurement on an entry route of the monitoring target, quarantine measures can be preemptively performed when a symptomatic person or a suspected case enters a country.

In this case, the beacon scanner 250 may receive body temperature information of a monitoring target from the patch-type thermometer 100 through short-range wireless communication such as near field communication (NFC) or Bluetooth. Of course, Bluetooth includes Bluetooth low energy (BLE) technology in addition to general Bluetooth technology. In addition, the beacon scanner 250 may transmit body temperature information received from the patch-type thermometer 100 to the web service server 200.

Meanwhile, the beacon scanner 250 may be installed in an inspection station as well as an immigration quarantine desk. As an example, the beacon scanner 250 may be installed in a drive-through inspection station. As another example, the beacon scanner 250 may be installed at an entrance of a medical facility such as a large hospital.

Thus, in the remote body temperature monitoring system 10 according to the embodiment of the present invention, since there is no need to be in close contact with a monitoring target in order to measure a body temperature, it is possible to prevent direct contact with the monitoring target, thereby ensuring the safety of quarantine even when the monitoring target is infected.

The echo device 400 may interwork with the AI server 300 through wired/wireless communication, and voice recognition is possible. The echo device 400 may recognize the voice of a user to transmit a command or request to the AI server 300 and may receive an analysis result of the AI server 300 to output the received analysis result as a voice. That is, the echo device 400 may be an input/output device of a user for the AI server 300.

As an example, the echo device 400 may output health check guide information provided by the AI server 300 as a voice according to collected body temperature information of a monitoring target.

The echo device 400 may receive data periodically transmitted from the patch-type thermometer 100. In this case, the echo device 400 may receive data from the patch-type thermometer 100 through short-range wireless communication such as NFC or Bluetooth. For example, the echo device 400 may receive body temperature information transmitted from the patch-type thermometer 100 through a BLE advertising method.

The echo device 400 may upload data received from the patch-type thermometer 100 to the web service server 200. Here, the echo device 400 may interwork with the web service server 200 related to the AI server 300 through wired/wireless communication.

As an example, the echo device 400 may be provided in a quarantine facility for a monitoring target. As another example, the echo device 400 may be provided in a self-quarantine facility for a monitoring target. In this case, an echo device provided at home may be set to interwork with the remote body temperature monitoring system 10.

Thus, through the remote body temperature monitoring system 10 according to the embodiment of the present invention, when a monitoring target is being self-quarantined or is residing in a living space of the monitoring target, in particular, a quarantine facility, since a body temperature of the monitoring target can be automatically measured and collected without special attention, the reliability and convenience in body temperature management can be improved.

The portable terminal 600 may be Bluetooth-paired with the patch-type thermometer 100 to receive body temperature information measured from the patch-type thermometer 100. Here, the portable terminal 600 may be an electronic device of a monitoring target, which is capable of performing wireless communication. As an example, the portable terminal 600 may be a smartphone, a smartpad, a personal computer (PC), a tablet PC, a netbook, or a notebook. In this case, the portable terminal 600 may include a body temperature management application.

The body temperature management application may perform Bluetooth pairing between the patch-type thermometer 100 and the portable terminal 600. In this case, the body temperature management application may periodically request and receive data from the patch-type thermometer 100 in a state in which the patch-type thermometer 100 and the portable terminal 600 are paired. Of course, corresponding data may also be transmitted from the patch-type thermometer 100 to the portable terminal 600 through tagging according to an NFC method.

Also, the portable terminal 600 may display a result value measured by the patch-type thermometer 100 through the body temperature management application. To this end, the body temperature management application may graphically provide body temperature information as graphs and figures. Accordingly, a monitoring target can easily check his or her body temperature state and state change trend.

Also, the portable terminal 600 may upload body temperature information received from the patch-type thermometer 100 to the web service server 200. Here, the portable terminal 600 may communicatively interwork with the web service server 200. To this end, the body temperature management application may allow the web service server 200 and the portable terminal 600 to interwork with each other.

In addition, the portable terminal 600 may interwork with the manager terminal 500. In this case, the portable terminal 600 may make an inquiry about a health check guide to the portable terminal 600. As an example, the portable terminal 600 may inquire about measures to be taken according to quarantine regulations and health statuses.

The manager terminal 500 may provide a health check guide according to a notification from the web service server 200 to the portable terminal 600. Here, the manager terminal 500 may be a portable electronic device such as a smartphone, a smartpad, a PC, a tablet PC, a netbook, or a notebook and may be an electronic device used or owned by a quarantine manager. However, the manager terminal 500 may provide health check guide information directly to the portable terminal 600 as shown in FIG. 1 or may provide the health check guide information to the portable terminal 600 through the web service server 200 unlike that shown in FIG. 1.

Meanwhile, a monitoring target (user) may be a person who cannot use the portable terminal 600 (hereinafter referred to as an “incapable user”), such as an infant, an elderly person with dementia, a disabled person, or the like. In this case, the beacon scanner 250 or the echo device 400 excluding the portable terminal 600 may operate as a collection device.

As an example, a collection device such as the beacon scanner 250 or the echo device 400 may be installed in facilities such as a kindergarten, a senior citizen center, and a welfare center, which accommodate monitoring targets that are incapable users, and the corresponding collection device may collect body temperature information measured by the patch-type thermometer 100 of the monitoring target to transmit the collected body temperature information to the web service server 200.

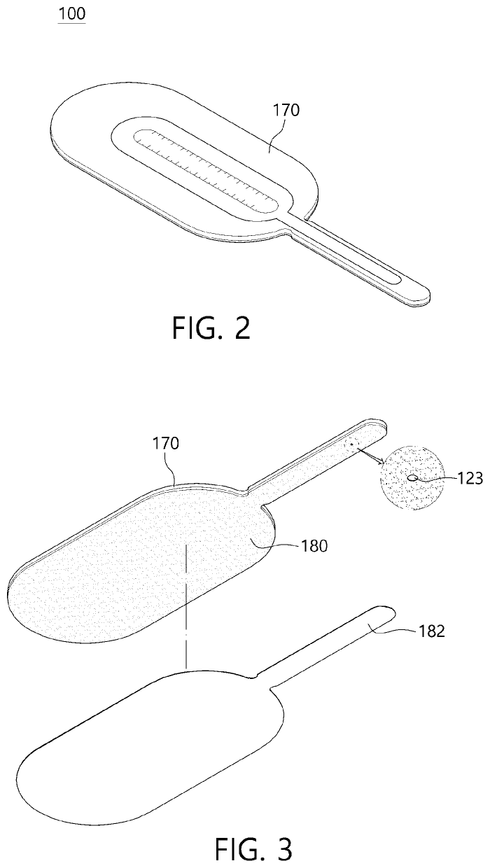

Referring to FIGS. 2 to 6, the patch-type thermometer 100 may include a flexible circuit board 110, one or more temperature sensors 121 and 122, a memory 130, a communicator 140, a controller 150, a power supply 160, and a protective member 170.

The flexible circuit board 110 may be a substrate on which various circuit elements are mounted or circuit patterns for electrical connection are formed. The flexible circuit board 110 may be a known flexible circuit board (FPCB) with flexibility which is made of polyimide (PI) or polyethylene terephthalate (PET).

The one or more temperature sensors 121 and 122 may be mounted on one surface of the flexible circuit board 110 to detect temperature information.

As an example, the one or more temperature sensors 121 and 122 may include a first temperature sensor 121 for detecting and measuring a body temperature of a user. The first temperature sensor 121 may be mounted on one surface of the flexible circuit board 110 and may measure a body temperature of a user at a certain time interval.

Here, information about the body temperature of the user measured by the first temperature sensor 121 may be stored in the memory 130. Thus, the patch-type thermometer 100 may record all changes in body temperature of the user over time.

In this case, the first temperature sensor 121 may be a digital temperature sensor and may directly measure a body temperature of a user but may measure the body temperature of the user based on heat transferred through a heat transfer member 123.

To this end, the first temperature sensor 121 may be mounted on a first surface of the flexible circuit board 110, and the heat transfer member 123 may be mounted on a second surface of the flexible circuit board 110 so as to be in direct contact with the user's skin. In this case, the first temperature sensor 121 and the heat transfer member 123 may be electrically connected to each other through a via hole 112 formed in the flexible circuit board 110. Here, the first surface and the second surface of the flexible circuit board 110 may be opposite to each other. As an example, the heat transfer member 123 may be made of a metal material having excellent thermal conductivity.

In this case, when the patch-type thermometer 100 is attached to user's skin, the heat transfer member 123 may have a shape capable of always maintaining a state of being in contact with the user's skin. To this end, the heat transfer member 123 may have a shape of which a central portion protrudes convexly in one direction. As an example, the heat transfer member 123 may be formed in a hemispherical shape or a dome shape.

For this reason, when the patch-type thermometer 100 is attached to the user's skin, even when an attachment part is a curved body part, the central portion of the heat transfer member 123 may always maintain a state of being in contact with the user' skin. As a result, the heat transfer member 123 may smoothly transfer heat transferred from the user's skin to the first temperature sensor 121.

As shown in FIGS. 3 and 5, the heat transfer member 123 may be exposed to the outside through an exposure hole 172 formed in the protective member 170 to be described below. Accordingly, when the patch-type thermometer 100 is attached to the user's body, the heat transfer member 123 may always be in direct contact with the user's skin.

In this case, the patch-type thermometer 100 may measure an external temperature as well as a body temperature of a user through the first temperature sensor 121. To this end, the patch-type thermometer 100 may further include a second temperature sensor 122 for measuring an external temperature in addition to the first temperature sensor 121 for measuring a body temperature of a user.

The second temperature sensor 122 may be mounted on one surface of the flexible circuit board 110 like the first temperature sensor 121 and may measure an external temperature at a certain time interval.

Thus, the patch-type thermometer 100 can measure information about a body temperature of a user through the first temperature sensor 121 as well as temperature information about an external environment at a time at which the body temperature of the user is measured so that the accuracy of the measured body temperature can be further enhanced.

As an example, the first and second temperature sensors 121 and 122 may be mounted on the same first surface of the flexible circuit board 110. That is, since the first surface is opposite to the second surface (surface in a direction in which the patch-type thermometer 100 is in contact with user's skin), temperature information measured by the second temperature sensor 122 may be reflected by minimizing an influence by a user and maximizing an influence by an external environment. In this case, temperature information about an external environment of the second temperature sensor 122, which is capable of serving as reference temperature information for deriving a body temperature of a user, may be measured more accurately. At the same time, the same influence by an external environment may act on the first and second temperature sensors 121 and 122. Accordingly, pieces of temperature information measured by the first and second temperature sensors 121 and 122 are used, and the temperature information measured by the first temperature sensor 121 is compared with the temperature information measured by the second temperature sensor 122, thereby further improving the accuracy of a finally derived body temperature of a user.

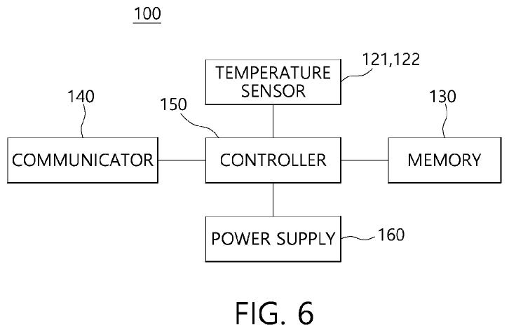

The overall operations of the one or more temperature sensors 121 and 122 may be controlled through the controller 150 as shown in FIG. 6, the controller 150 may be operated using power provided through the power supply 160, and information obtained through the one or more temperature sensors 121 and 122 may be stored in the memory 130. As an example, the controller 150 may include a processor that is hardware, a process that is software and is executed in the processor, and the like. The process may be stored in the memory 130.

In this case, the one or more temperature sensors 121 and 122 may periodically detect a temperature at a certain time interval. Thus, the patch-type thermometer 100 may periodically measure and store a body temperature of a user or an external temperature while minimizing consumption of power provided from the power supply 160 so that a battery replacement cycle or recharge cycle can be increased.

The communicator 140 may transmit body temperature information stored in the memory 130 to an external electronic device through a short-range wireless communication method. Here, the external electronic device may include the beacon scanner 250, the echo device 400, and the portable terminal 600.

As an example, the communicator 140 may include an NFC module. In this case, when the portable terminal 600 provided with an NFC module is tagged to the communicator 140, information stored in the memory 130 may be transmitted to the portable terminal 600.

To this end, the communicator 140 may include an antenna pattern 141 patterned and formed on at least one surface of the flexible circuit board 110 and a driving chip 142 mounted on one surface of the flexible circuit board 110 to drive the antenna pattern 141.

Accordingly, the antenna pattern 141 may serve as a radiator for transmitting information acquired through the one or more temperature sensors 121 and 122 to the outside using a short-range wireless communication method.

In this case, data transmitted to the portable terminal 600 through the antenna pattern 141 during NFC tagging may be displayed through the body temperature management application installed on the portable terminal 600. The body temperature management application may provide a trend of a change in body temperature.

In this case, the driving chip 142 may be disposed inside the antenna pattern 141, the first temperature sensor 121 may be disposed outside the antenna pattern 141, and the driving chip 142 and the temperature sensors 121 and 122 may be electrically connected to each other through a lead 143 formed on at least one surface of the flexible circuit board 110.

As another example, the communicator 140 may include a Bluetooth module. In this case, the communicator 140 may be Bluetooth-paired with the portable terminal 600 and may transmit temperature information measured through the temperature sensors 121 and 122 to the portable terminal 600.

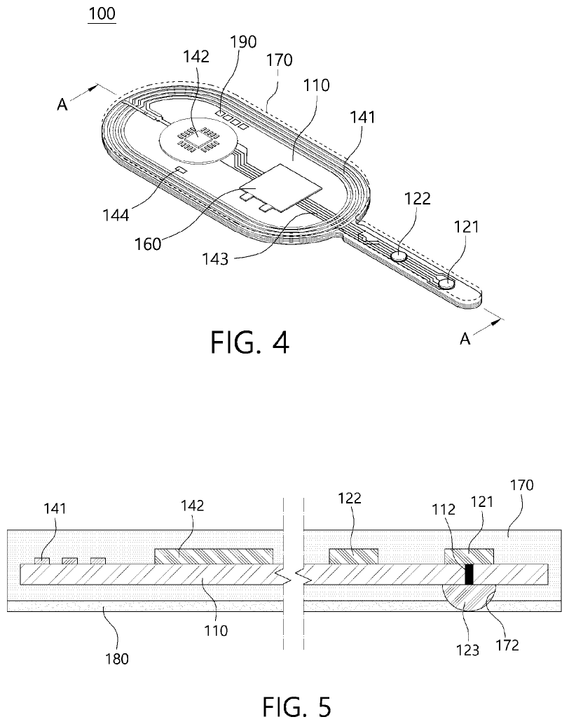

In addition, the communicator 140 may be formed in a form including both the NFC module (antenna pattern 141, driving chip 142, and lead 143) and a Bluetooth module 144 as shown in FIG. 4.

The controller 150 may control the overall operation of the patch-type thermometer 100 by controlling the temperature sensors 121 and 122, the memory 130, and the communicator 140. The controller 150 may be operated using power provided through the power supply 160.

In addition, the power supply 160 may be a known coin battery or a prismatic battery but may be a plate-shaped flexible battery so as to increase capacity while reducing the overall weight and volume thereof. As a specific example, the power supply 160 may be a known paper battery or a printed battery.

That is, the power supply 160 may be implemented as a plate-shaped flexible battery so that power capacity for operating the patch-type thermometer 100 can be sufficiently secured, the overall weight thereof can be reduced, and the patch-type thermometer 100 can be implemented to be thin.

Meanwhile, the patch-type thermometer 100 may include the protective member 170 configured to surround the flexible circuit board 110 so as to prevent the one or more temperature sensors 121 and 122, the memory 130, the communicator 140, the controller 150, and the power supply 160 from being exposed to the outside.

In this case, the protective member 170 may include the exposure hole 172 formed in a region corresponding to the heat transfer member 123 as described above, and the heat transfer member 123 may be exposed to the outside through the exposure hole 172.

Accordingly, the protective member 170 may be formed to completely cover the remaining portion other than the heat transfer member 123.

In this case, the protective member 170 may be made of a material having flexibility. Thus, even when the patch-type thermometer 100 is attached to a curved body part, the patch-type thermometer 100 can be flexibly changed according to a body curve of a user, thereby increasing adhesion to the user's body.

As an example, the protective member 170 may be a molded body made of an insulating resin material such as silicone. However, the present invention is not limited thereto, and the protective member 170 may be in the form of a sheet made of release paper or a fluoropolymer resin such as PET, polypropylene (PP), or polyethylene (PE). Any material having an insulating property and airtightness may be used without limitation.

Meanwhile, the patch-type thermometer 100 may include an adhesive member 180 disposed on one surface of the protective member 170.

The adhesive member 180 may impart adhesion or an adhesive force to attach the patch-type thermometer 100 to a user's body. Here, the adhesive member 180 may be disposed on a surface in which the exposure hole 172 for exposing the heat transfer member 123 to the outside is formed.

Accordingly, when the patch-type thermometer 100 is attached to the user's skin through the adhesive member 180, the heat transfer member 123 may be in direct contact with the user's skin.

As an example, the adhesive member 180 may be a gel-type adhesive layer and may be made of a material whose adhesive force is restored upon contact with moisture. Accordingly, the adhesive member 180 may be repeatedly reused. As another example, the adhesive member 180 may be a known double-sided tape.

In addition, a removable release film 182 may be additionally attached to an exposed surface of the adhesive member 180.

Meanwhile, the patch-type thermometer 100 may further include at least one indicator 190.

The indicator 190 may display a remaining battery level of the power supply 160 or an on/off operating state. As an example, the indicator 190 may be at least one light-emitting diode (LED) mounted on one surface of the flexible circuit board 110.

Although embodiments of the present embodiment have been described, the spirit of the present invention is not restricted to the embodiments described herein, and it shall be appreciated that those having ordinary skill in the art who understand the present invention can propose other embodiments by modifying, deleting, and adding elements within the scope of the same spirit, and this shall also fall within the scope of the present invention.

### Industrial Applicability

The present invention relates to a remote body temperature monitoring system. The remote body temperature monitoring system can periodically and remotely measure a body temperature of a monitoring target for a certain period of time, and thus has industrial applicability.

1. A remote body temperature monitoring system comprising:
a patch-type thermometer attached to a user's body to periodically measure a body temperature;
a web service server configured to collect body temperature information measured by the patch-type thermometer and provide a notification to a manager terminal when a fever is suspected;
a collection device configured to receive the measured body temperature information from the patch-type thermometer and transmit the received body temperature information to the web service server; and
an artificial intelligence (AI) server configured to receive the body temperature information from the web service server and manage a health status through AI.

2. The remote body temperature monitoring system of claim 1, wherein:
the collection device includes an echo device configured to communicatively interwork with the AI server and perform voice recognition;
the patch-type thermometer periodically transmits data; and
the echo device uploads the data received from the patch-type thermometer to the web service server.

3. The remote body temperature monitoring system of claim 2, wherein:
the AI server provides health check guide information according to heat detection to the echo device; and
the echo device outputs the health check guide information as a voice.

4. The remote body temperature monitoring system of claim 1, wherein the collection device includes a beacon scanner installed at an entrance of a specific area.

5. The remote body temperature monitoring system of claim 4, wherein the specific area is a quarantine desk of an airport and a port.

6. The remote body temperature monitoring system of claim 1, wherein the collection device includes a portable terminal which is Bluetooth-paired with the patch-type thermometer to receive the measured body temperature information and on which an application is installed.

7. The remote body temperature monitoring system of claim 6, wherein:
the manager terminal provides a health check guide according to the notification from the web service server to the portable terminal; and
the portable terminal makes an inquiry about the health check guide to the manager terminal.

8. The remote body temperature monitoring system of claim 1, wherein the web service server notifies the manager terminal through a push message or a short message service (SMS) message.

9. The remote body temperature monitoring system of claim 1, wherein the patch-type thermometer includes:
at least one temperature sensor mounted on one surface of a flexible circuit board;
a memory configured to store information obtained through the temperature sensor;
a communicator configured to transmit the information stored in the memory to an external device;
a controller configured to control operation of the temperature sensor, the memory, and the communicator;
a power supply disposed on one surface of the flexible circuit board to provide operating power; and
a protective member configured to surround the flexible circuit board to prevent external exposure of the temperature sensor, the memory, the communicator, the controller, and the power supply.
